(19) United States
(12) Patent Application Publication
(10) Pub. No.: US 2019/0274557 A1
(43) Pub. Date: Sep. 12, 2019

(54) Blood Pressure Detection Device

(71) Applicant: Zhongshan Anbo Health Technology Co., Ltd., Zhongshan (CN)

(72) Inventor: Wenhui ZHOU, Zhongshan (CN)

(73) Assignee: Zhongshan Anbo Health Technology Co., Ltd., Zhongshan (CN)

(21) Appl. No.: 15/916,206

(22) Filed: Mar. 8, 2018

(51) Int. Cl. A61B 5/022; A61B 5/0235; A61B 5/00

(52) U.S. Cl. CPC A61B 5/02233; A61B 5/02241; A61B 5/6891; A61B 5/6844; A61B 5/0235

(57) ABSTRACT

A blood pressure detection device, includes a detection airbag, a pressure sensor provided on the detection airbag, an air pump configured for inflating detection airbag and a solenoid valve or a proportional valve or an exhaust valve for deflating detection airbag. The blood pressure detection device further includes an airbag clamp configured outside the detection airbag for clamping the inflated detection airbag on the finger, wrist, or arm of the human body. The present invention eliminates the operation of wrapping the airbag on the arm or the wrist in the traditional blood pressure detection, so that the blood pressure detection is more convenient. In addition, the present invention incorporates an airbag into the device of the present invention, so as to avoid separation of the airbag from the detection device body, thereby facilitating the combination of the present invention with household or sanitary products.

### TECHNICAL FILED

The present invention relates to a blood pressure detection device.

### BACKGROUND OF THE INVENTION

Traditional blood pressure detection mainly uses a ring-shaped airbag to wind and wrap around a human wrist or arm, and then the airbag is inflated so that the pressure sensor in the airbag is attached close to the blood vessel of the human and the blood pressure is detected. Therefore, all the blood pressure detection products in the market include a host and an airbag connected to the host through a tube. When using these traditional blood pressure detection products, user has to tie the airbag to the arm, which is troublesome to operate. If the airbag is accidentally lost, then the entire product will be useless. In addition, if the blood pressure detection device is required to be integrated into daily life and combined with household or sanitary products, the separately connected airbag would affect the appearance and daily usage.

### SUMMARY OF THE INVENTION

In order to solve the deficiencies of the prior art, the present invention provides a blood pressure detection device that eliminates the requirement of wrapping the airbag on the arm or the wrist in the traditional blood pressure detection, so that the blood pressure detection is more convenient. In addition, the present invention incorporates an airbag into the device of the present invention, so as to avoid separation of the airbag from the detection device body, thereby facilitating the combination of the present invention with household or sanitary products. Further, the present invention can adapt to the finger or wrist or arm blood pressure detection by changing the size of the structure.

The technical solutions adopted by the present invention are provided as below:

A human blood pressure detection device, comprises detection airbag 1 which forms a closed or a non-closed ring after being inflated when detecting the blood pressure, a pressure sensor provided on detection airbag 1, an air pump configured for inflating detection airbag 1 and a solenoid valve or a proportional valve or an exhaust valve for deflating detection airbag 1; the human blood pressure detection device further comprises airbag clamp 2 configured outside the detection airbag for clamping inflated detection airbag 1 on the finger, wrist, or arm of the human body.

Airbag clamp 2 includes supporting portion 20 and clamping portion 21 provided on supporting portion 20. The supporting portion, the clamping portion or both the supporting portion and the clamping portion are provided with or formed as detection ring 23 for accommodating a human finger or a wrist or an arm. The detection ring 23 is provided with detection inlet 24 for allowing or restricting a human finger or a wrist or an arm to enter into or leave from detection ring 23. Detection airbag 1 is provided on detection ring 23. Airbag clamp 2 further includes driving device 22, wherein driving device 22 drives clamping portion 21 to move relative to supporting portion 20, such that detection inlet 24 is expanded or narrowed to allow the human finger or the wrist or the arm to enter into or leave from detection ring 23.

Airbag clamp 2 further comprises the supporting portion, the clamping portion arranged on the supporting portion, and a driving device. The driving device drives the clamping portion to move relative to the supporting portion, such that the clamping portion or the supporting portion and the clamping portion form a closed or non-closed ring, which surrounds the human finger or the wrist or the arm; the detection airbag is provided on the clamping portion and/or the supporting portion.

Clamping portion 21 comprises first clamping portion 211 and second clamping portion 212; free end 213 and driving end 214 are provided on each of first clamping portion 211 and second clamping portion 212. Driving ends 214 of first clamping portion 211 and second clamping portion 212 are connected to driving device 22. Free ends 213 of first clamping portion 211 and second clamping portion 212 move close or away from each other when first clamping portion 211 and second clamping portion 212 are driven by driving device 22 to move relative to the supporting portion, so that detection inlet 24 is narrowed or expanded.

Detection airbag 1 includes airbag body 10 and connecting ends 11 located at two ends of airbag body 10, wherein connecting ends 11 are connected to free ends 213 of first clamping portion 211 and second clamping portion 212.

First clamping portion 211 and second clamping portion 212 are arc-shaped, supporting portion 20 includes supporting portion body 200. Wherein supporting portion body 200 is U-shaped and works as detection ring 23.

Free end 213 of first clamping portion 211 is provided with a protrusion, and free end 213 of second clamping portion 212 is provided with a groove fitted with the protrusion provided on free end 213 of first clamping portion 211.

Sliding grooves 201 are provided on supporting portion 20, and first clamping portion 211 and second clamping portion 212 are each provided with sliding portion 215 for engaging with sliding grooves 201. Driving device 22 includes first driving rod 221 and second driving rod 222 respectively hinged with driving ends 214 of first clamping portion 211 and second clamping portion 212, movable driving block 223 hinged with the other ends of first driving rod 221 and second driving rod 22, driving airbag 224 provided between movable driving block 223 and supporting portion 20 to push movable driving block 223 to move, an air pump used for inflating driving airbag 224, a solenoid valve or a proportional valve or an exhaust valve configured for deflating driving airbag 224. Driving device 22 includes a reset spring provided between supporting portion 20 and movable driving block 223 to push movable driving block 223 to reset; the movable driving block is located between the driving airbag and the reset spring.

Airbag clamp 2 further comprises hand sensing module 23, wherein the hand sensing module is used to detect whether the human finger, the wrist, or the arm is located in detection ring 23 or on supporting portion 20.

The hand sensing module comprises a capacitive sensor or a pressure sensor, a light touch switch, a sensing electrode plate, a button switch, or an infrared sensor provided on supporting portion 20 or clamping portion 21 or detection airbag 1.

Compared with the prior art, the present invention has advantages as below:

1. In the present invention, the inflated airbag is pressed against the finger, the wrist or the arm of the human body by the clamping function of the airbag clamp, so that the user no longer needs to tie the detection airbag to the finger or the wrist or the arm, and the blood pressure detection is convenient. In addition, the present invention incorporates the detection airbag into the device of the present invention to facilitate the combination of the present invention with household or sanitary products. Certainly, the present invention can form a complete blood pressure detection product by adding control circuit, power supply circuit and a housing. Besides, the present invention has the advantage of adaptability by changing the size of the structure, so as to adapt to the finger, wrist, or arm blood pressure detection.

2. As a further improvement of the present invention, the airbag clamp includes a supporting portion, a clamping portion, and a driving device. The supporting portion, the clamping portion, or both the supporting portion and the clamping portion are provided with or formed as a detection ring for accommodating a finger, a wrist, or an arm of human. The detection ring is provided with a detection inlet for allowing or restricting the human finger or the wrist or the arm to enter into or leave from the detection ring, and the detection airbag is arranged on the detection ring. When the user measures the blood pressure, the finger or the wrist or the arm is first placed on the detection ring through the detection inlet and the driving device drives the clamping portion to move relative to the supporting portion, so that the detection inlet is narrowed or directly closed and the human finger or the wrist or the arm is surrounded in the detection ring, then the blood pressure detection can start. After detecting the blood pressure, the controller controls the driving device to expand the detection inlet, so that the user can remove his/her finger, wrist, or arm. The airbag clamp is provided with a detection ring, which is convenient for the user to place a finger or a wrist or an arm and makes the blood pressure detection more convenient.

### BRIEF DESCRIPTION OF THE DRAWINGS

FIG. 1 is a schematic diagram of perspective view of the present invention;

FIG. 2 is a schematic diagram, in which the blood pressure detection device of the present invention is used on the toilet.

### DETAILED DESCRIPTION OF THE INVENTION

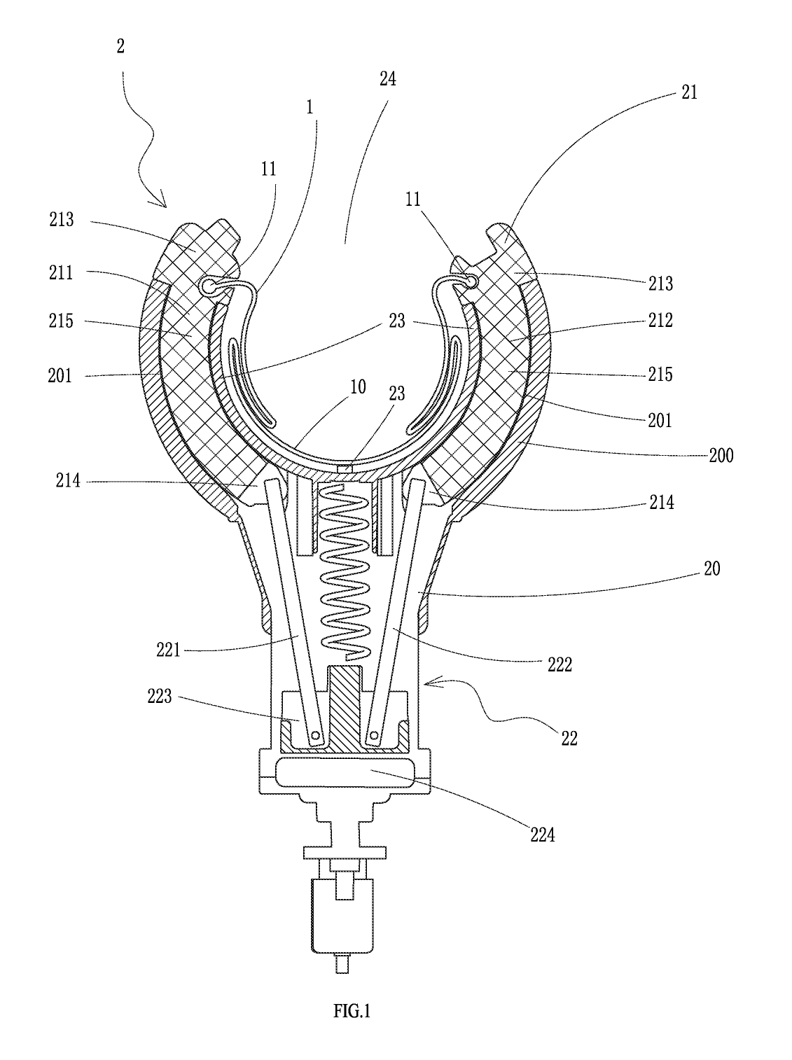

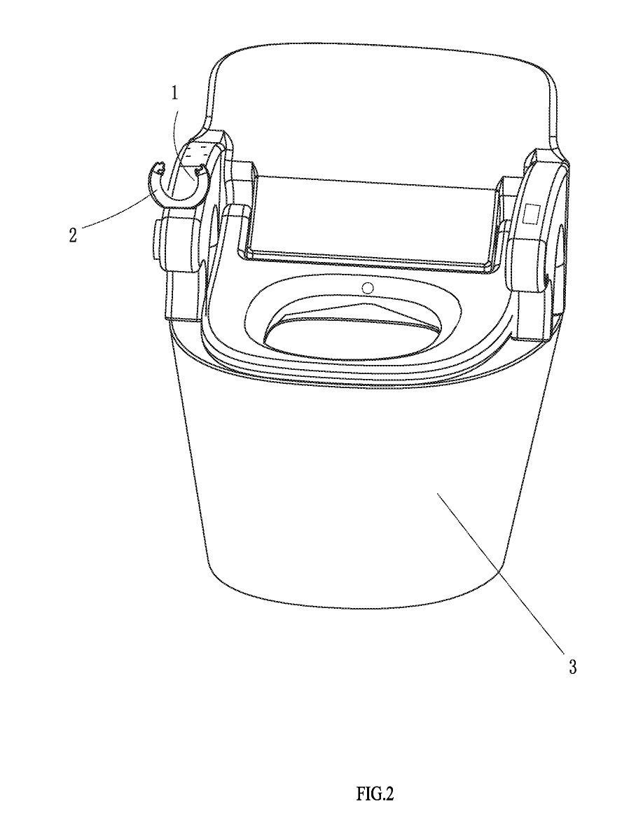

The embodiments of the present invention will be described in detail with reference to FIG. 1 and FIG. 2.

A blood pressure detection device, comprises inflated detection airbag 1 which is closed or non-closed ring shape when detecting the blood pressure, a pressure sensor provided on detection airbag 1, an air pump configured for inflating detection airbag 1 and a solenoid valve or a proportional valve or an exhaust valve for deflating detection airbag 1. The blood pressure detection device further comprises airbag clamp 2 arranged outside the detection airbag and configured for clamping the inflated detection airbag 1 on the finger, wrist, or arm of the human body. The airbag clamp can open or close to allow or restrict the human finger, the wrist, or the arm to enter into or leave from the airbag clamp. The inflated detection airbag 1 is tightly attached to the finger, wrist, or arm of the human body by airbag clamp 2, so that the pressure sensor on detection airbag 1 is tightly attached to the blood vessels of the finger, wrist, or arm of the human body. When detecting the blood pressure, the user puts his or her finger, wrist, or arm in airbag clamp 2, such that detection airbag 1 is located between the finger, wrist, or arm and the airbag clamp 2. Then the air pump begins to inflate detection airbag 1 and the finger, wrist, or arm is pressed tightly through the clamping function of airbag clamp 2. The detection airbag 1 is inflated until the pressure of detection airbag 1 applied to the finger, wrist or arm in airbag clamp 2 is enough to stop the blood flow. And then, the solenoid valve, the proportional valve, or the exhaust valve evenly deflates detection airbag 1. The pressure sensor obtains the pressure fluctuation data related to the human blood pressure from the above process and converts the data into electrical signals. The electrical signals are transmitted to the additionally provided controller. The controller obtains the human blood pressure value according to the oscillometric method of blood pressure detection and the obtained blood pressure value of the human body is output through output modules such as display module, voice module, or communication module which communicates with the smart device.

The method for detecting blood pressure based on the oscillometric method is as follows: In the above oscillatory wave data, the time of the largest fluctuation is selected as the reference point. On the basis of the reference point, a fluctuation point of 0.45 times the peak is searched forward, and this fluctuation point corresponds to the systolic blood pressure; a fluctuation point of 0.75 times the peak is searched backward, and this fluctuation point corresponds to the diastolic blood pressure. It should be noted that the constants of 0.45 and 0.75 vary from person to person and should be set based on the results of clinical tests. Moreover, different blood pressures may be segmented and set with different constants accordingly.

### EMBODIMENT

Airbag clamp 2 includes supporting portion 20 and clamping portion 21 provided on support portion 20, wherein the supporting portion, the clamping portion, or both the supporting portion and the clamping portion are provided with or formed as detection ring 23 for accommodating a human finger or a wrist or an arm. Detection ring 23 is provided with detection inlet 24 for allowing or restricting the human finger or the wrist or the arm to enter into or leave from detection ring 23. Detection airbag 1 is provided on detection ring 23. Airbag clamp 2 further includes driving device 22, wherein driving device 22 drives clamping portion 21 to move relative to supporting portion 20, such that detection inlet 24 is expanded or contracted to allow the human finger or the wrist or the arm to enter into or leave from detection ring 23. Detection ring 23 can be provided on supporting portion 20, and the shape of the supporting portion forms the detection ring. Detection ring 23 can also be provided on clamping portion 21, and the shape of clamping portion 21 forms the detection ring. Further, detection ring 23 can be formed by the supporting portion and clamping portion together. The finger, wrist, or arm of the human body entering into the detection ring means that the finger, wrist or arm of the human body enters into the detection ring, and is surrounded by the detection ring.

When detecting the blood pressure, first, the user places a finger, wrist, or arm into detection ring 23. Driving device 22 drives clamping portion 21 to move relative to supporting portion 20, so that detection inlet 24 on detection ring 23 is closed or narrowed, whereby the detection ring encloses the human finger, wrist, or arm and restrains the finger, wrist, or arm of the human body from leaving the detection ring through the detection inlet. Meanwhile, detection airbag 1 is located between the human finger or wrist or arm and the detection ring. When inflating detection airbag 1, detection airbag 1 is tightly pressed between the detection ring and the human finger or wrist or arm, and the pressure sensor on detection airbag 1 is attached close to the blood vessels of the human body, thereby obtaining the accurate data. Preferably, detection inlet 24 is closed when detecting the blood pressure, so that the entire detection ring forms a complete ring with better strength when detecting the blood pressure. However, if the clamping portion and the supporting portion have sufficient strength and rigidity, the detection inlet is also allowed to be opened and just be narrowed to the degree that the finger, the wrist, or the arm of the human body cannot be easily removed from the detection ring when the blood pressure detection is performed.

Certainly, the following preferable design may also be made: Airbag clamp 2 includes a supporting portion, a clamping portion provided on the supporting portion, and a driving device, wherein the driving device drives the clamping portion to move relative to the supporting portion, such that the clamping portion or the supporting portion and the clamping portion form a closed or non-closed ring surrounding the finger, wrist or arm of the human body. The detection airbag is provided on the clamping portion and/or the supporting portion. Airbag clamp 2 can be provided without a fixed detection ring. When the user detects the blood pressure, first, the finger or the wrist or the arm is placed on the supporting portion, and the driving device drives the clamping portion to move relative to the supporting portion, such that the clamping portion or both the supporting portion and the clamping portion form a closed or non-closed ring surrounding the human finger or wrist or arm. The closed or non-closed ring is equivalent to the detection ring, and is formed when detecting the blood pressure, instead of being set at the beginning. Detection airbag 1 is already located between the human finger, the wrist, or the arm and the airbag clamp when detecting the blood pressure, and when inflating the detection airbag, the detection airbag is tightly pressed between the detection ring and the human finger or wrist or arm. After the blood pressure measurement is completed, the driving device drives the clamping portion to move relative to the supporting portion so that the position of the clamping portion is reset, and the user can remove the finger, the wrist, or the arm. This design is beneficial to hide the clamping portion of airbag clamp 2 in the supporting portion, and the clamping portion of airbag clamp 2 is exposed from the supporting portion to form the detection ring when detecting the blood pressure, making the appearance of the present invention more harmonious. Further, in the preferred embodiment, a hand sensing module for sensing whether the human finger, the wrist, or the arm is on the supporting portion can be provided on the supporting portion. The hand sensing module is preferably a capacitive sensor, a pressure sensor, an infrared sensor, a sensing electrode plate, or a button switch. After the human finger or the wrist or the arm is placed on the supporting portion, the hand sensing module senses the human finger or the wrist or the arm and then notifies the driving device to drive the clamping portion to move relative to the supporting portion and form a closed or non-closed ring surrounding the human finger, wrist, or arm.

For symmetry and considerations of maintaining the stiffness and strength, clamping portion 21 comprises first clamping portion 211 and second clamping portion 212. Free end 213 and driving end 214 are provided on each of first clamping portion 211 and second clamping portion 212. Driving ends 214 of first clamping portion 211 and second clamping portion 212 are connected to driving device 22. Free ends 213 of first clamping portion 211 and second clamping portion 212 are moved close to or away from each other when first clamping portion 211 and second clamping portion 212 are driven to move relative to the supporting portion by driving device 22, so that detection inlet 24 is expanded or narrowed. Detection inlet 24 is formed between free ends 213 of first clamping portion 211 and second clamping portion 212. When free ends 213 of first clamping portion 211 and second clamping portion 212 move close to each other, detection inlet 24 is narrowed or even closed; and when free ends 213 of first clamping portion 211 and second clamping portion 212 move away from each other, detection inlet 24 is opened or expanded.

Detection airbag 1 includes airbag body 10 and connecting portions 11 located at two ends of airbag body 10, wherein connecting portions 11 are connected to free ends 213 of first clamping portion 211 and second clamping portion 212. The two ends of detection airbag 1 are respectively connected to free ends 213 of first clamping portion 211 and second clamping portion 212. When the human finger, wrist, or arm is placed in the detection ring, and free ends 213 of first clamping portion 211 and second clamping portion 212 move close to each other, detection airbag 1 opens and encloses the human finger, wrist, or arm.

For fitting the shape of the human finger or wrist or arm to improve the comfort in blood pressure detection, first clamping portion 211 and second clamping portion 212 are arc-shaped. Supporting portion 20 includes supporting portion body 200; wherein supporting portion body 200 is U-shaped and works as detection ring 23.

Free end 213 of first clamping portion 211 is provided with a protrusion, and free end 213 of second clamping portion 212 is provided with a groove fitted with the protrusion provided on free end 213 of first clamping portion 211. After free ends 213 of first clamping portion 211 and second clamping portion 212 move close to each other, the above-mentioned protrusion and groove cooperate with each other, so that first clamping portion 211, second clamping portion 212 and supporting portion 20 together form a stable closed ring. When inflating detection airbag 1, the strength and stiffness of airbag clamp 2 can also be ensured, so as to ensure the accuracy of blood pressure detection results.

Sliding grooves 201 are provided on supporting portion 20, and first clamping portion 211 and second clamping portion 212 are provided with sliding portions 215 engaged with sliding grooves 201. Driving device 22 includes first driving rod 221 and second driving rod 222, respectively hinged with driving ends 214 of first clamping portion 211 and second clamping portion 212, movable driving blocks 223 hinged with the other ends of first driving rod 221 and second driving rod 222, driving airbag 224 provided between movable driving block 223 and supporting portion 20 and used to push movable driving block 223 to move, an air pump used for inflating driving airbag 224, a solenoid valve or a proportional valve or an exhaust valve for deflating driving airbag 224. Driving device 22 further includes a reset spring provided between supporting portion 20 and movable driving block 223 and used to push movable driving block 223 to reset. The movable driving block is located between the driving airbag and the reset spring. The solenoid valve, the proportional valve, or the exhaust valve may also be replaced with other devices that can exhaust the driving airbag. Such replacement is a replacement of conventional technical means and should fall within the protection scope of the present invention.

When the user places a finger, wrist, or arm in detection ring 23 through detection inlet 24, the air pump inflates the driving airbag. The volume of the driving airbag becomes larger and drives the movable driving block to move. The movable driving block pushes first driving rod 221 and second driving rod 222 to move. First driving rod 221 and second driving rod 222 respectively push first clamping portion 211 and second clamping portion 212 to slide in sliding grooves 201 of the supporting portion, so that the free ends of first clamping portion 211 and second clamping portion 212 move closer to each other, therefore detection inlet 24 is closed or narrowed. The closed or narrowed detection inlet limits the movement of the human finger, wrist, or arm, so that the blood pressure detection can be performed. After the blood pressure detection is completed, the solenoid valve, the proportional valve, or the exhaust valve deflates the driving airbag, and the movable driving block is reset by the reset spring and drives the driving rods and first clamping portion 211 and second clamping portion 212 to reset at the same time. Detection inlet 24 is opened or expanded again, so that the human finger, the wrist, or the arm can be removed from detection ring 23. The power source of the driving device in this embodiment is an air pump, which adopts the pneumatic driving mode. Since the gas gets easily compressed, the airbag clamp will not cause any big harm to the user even if a situation of clamping hand occurs, which has an advantage of being safe.

Airbag clamp 2 further includes a hand sensing module, wherein the hand sensing module is used to detect whether the human finger, the wrist, or the arm is located in detection ring 23 or on supporting portion 20. The human body sensing module includes a capacitive sensor or a pressure sensor, a light touch switch, a sensing electrode plate, a button switch, or an infrared sensor provided on supporting portion 20 or clamping portion 21 or detection airbag 1.When the user places a finger or wrist or arm in detection ring 23 or on supporting portion 20, the hand sensing module informs the additionally provided controller that the user is in place, wherein the controller is connected to the hand sensing module. The controller controls driving device 22 to drive clamping portion 21 to move relative to supporting portion 20, so that detection inlet 24 is narrowed or even closed, and the blood pressure detection gets started.

The present invention can be combined with household or sanitary products to incorporate blood pressure detection into people's daily lives. FIG. 2 is a perspective view of the present invention when the present invention is applied to toilet 3. The toilet having the blood pressure detection device enables users to pay attention to their own blood pressure status while they are using the toilet every day, so as to know their own health status.

What is claimed is:

1. A human blood pressure detection device, comprising:
a detection airbag, wherein the detection bag forms a closed or a non-closed ring after being inflated when detecting the blood pressure,
a pressure sensor provided on the detection airbag,
an air pump configured for inflating the detection airbag,
a solenoid valve or a proportional valve or an exhaust valve for deflating the detection airbag; and
an airbag clamp provided outside the detection airbag and configured for clamping the inflated detection airbag on the finger, wrist, or arm of the human body.

2. The human blood pressure detection device according to claim 1, wherein the airbag clamp includes a supporting portion and a clamping portion provided on the supporting portion; the supporting portion, the clamping portion or both the supporting portion and the clamping portion are provided with or formed as a detection ring for accommodating a human finger or a wrist or an arm; the detection ring is provided with a detection inlet for allowing or restricting the human finger or the wrist or the arm to enter into or leave from the detection ring; the detection airbag is provided on the detection ring; the airbag clamp further includes a driving device, wherein the driving device drives the clamping portion to move relative to the supporting portion, such that the detection inlet is expanded or narrowed to allow the human finger or the wrist or the arm to enter into or leave from the detection ring.

3. The human blood pressure detection device according to claim 1, wherein the airbag clamp further comprises a supporting portion, a clamping portion arranged on the supporting portion, a driving device, wherein the driving device drives the clamping portion to move relative to the supporting portion, such that the clamping portion or both the supporting portion and the clamping portion form a closed or non-closed ring configured to surround a human finger or a wrist or an arm; the detection airbag is provided on the clamping portion and/or the supporting portion.

4. The human blood pressure detection device according to claim 2, wherein the clamping portion comprises a first clamping portion and a second clamping portion; a free end and a driving end are provided on each of the first clamping portion and the second clamping portion; the driving ends of the first clamping portion and the second clamping portion are connected to the driving device; the free ends of the first clamping portion and the second clamping portion move close or away from each other when the first clamping portion and the second clamping portion are driven by the driving device to move relative to the supporting portion, so that the detection inlet is narrowed or expanded.

5. The human blood pressure detection device according to claim 4, wherein the detection airbag includes an airbag body and connecting ends located at two ends of the airbag body, wherein the connecting ends are connected to the free ends of the first clamping portion and the second clamping portion.

6. The human blood pressure detection device according to claim 4, wherein the first clamping portion and the second clamping portion are arc-shaped; the supporting portion includes a supporting portion body; wherein the supporting portion body is U-shaped and works as the detection ring.

7. The human blood pressure detection device according to claim 4, wherein the free end of the first clamping portion is provided with a protrusion, and the free end of the second clamping portion is provided with a groove fitted with the protrusion provided on the free end of the first clamping portion.

8. The human blood pressure detection device according to claim 4, wherein sliding grooves are provided on the supporting portion; and the first clamping portion and the second clamping portion are each provided with a sliding portion for engaging with the sliding grooves; the driving device includes the first driving rod and the second driving rod respectively hinged with driving ends of the first clamping portion and the second clamping portion, a movable driving block hinged with the other ends of the first driving rod and the second driving rod, a driving airbag provided between the movable driving block and the supporting portion to push the movable driving block to move, an air pump used for inflating the driving airbag, a solenoid valve or a proportional valve or an exhaust valve configured for deflating the driving airbag;
wherein the driving device includes a reset spring provided between the supporting portion and the movable driving block to push the movable driving block to reset; the movable driving block is located between the driving airbag and the reset spring.

9. The human blood pressure detection device according to claim 2, wherein the airbag clamp further comprises a hand sensing module, wherein the hand sensing module is used to detect whether the human finger, the wrist, or the arm is located in the detection ring or on the supporting portion.

10. The human blood pressure detection device according to claim 9, wherein the hand sensing module comprises a capacitive sensor or a pressure sensor or a light touch switch or a sensing electrode plate or a button switch or an infrared sensor provided on the supporting portion or the clamping portion or the detection airbag.

11. The human blood pressure detection device according to claims 3, wherein the airbag clamp further comprises a hand sensing module, wherein the hand sensing module is used to detect whether the human finger, the wrist, or the arm is located in the detection ring or on the supporting portion.

12. The human blood pressure detection device according to claim 11, wherein the hand sensing module comprises a capacitive sensor or a pressure sensor or a light touch switch or a sensing electrode plate or a button switch or an infrared sensor provided on the supporting portion or the clamping portion or the detection airbag.
